(19) United States
(12) Patent Application Publication
(10) Pub. No.: US 2019/0365837 A1
(43) Pub. Date: Dec. 5, 2019

(54) ANTI-NASH COMPOSITION, FOOD COMPOSITION FOR PREVENTING NASH, BEVERAGE COMPOSITION FOR PREVENTING NASH, COMPOSITION FOR PREVENTING CIRRHOSIS, AND COMPOSITION FOR PREVENTING HEPATOCELLULAR CARCINOMA

(71) Applicant: MYCOLOGY TECHNO. CORP., Niigata-shi, Niigata (JP)

(72) Inventors: Kenichi WATANABE, Niigata-shi, Niigata (JP); Tetsuya KONISHI, Niigata-shi, Niigata (JP); Yusuke KOGA, Niigata-shi, Niigata (JP)

(21) Appl. No.: 16/484,204

(22) PCT Filed: Feb. 6, 2018
(86) PCT No.: PCT/JP2018/003958

§ 371 Date: Aug. 7, 2019

(30) Foreign Application Priority Data
Feb. 7, 2017 (JP) 2017-020741

(51) Int. Cl. A61K 36/07; A23L 2/38; A61P 1/16; A23L 33/105

(52) U.S. Cl. CPC A61K 36/07; A23L 33/105; A61P 1/16; A23L 2/38

(57) ABSTRACT

This anti-NASH composition, this food composition for preventing NASH, and this beverage composition for preventing NASH include, as an active ingredient, a Basidiomycetes-X FERM BP-10011 dry powder or an extract composition thereof. In addition, a composition for preventing cirrhosis and a composition for preventing hepatocellular carcinoma include, as an active ingredient, a Basidiomycetes-X FERM BP-10011 dry powder or an extract composition thereof, and prevent metastasis of cirrhosis and hepatocellular carcinoma from NASH.

### TECHNICAL FIELD

The present invention relates to an anti-NASH composition, to a food composition for preventing NASH (hereinafter may be referred to as a “NASH-preventing food composition”), to a beverage composition for preventing NASH (hereinafter may be referred to as a “NASH-preventing beverage composition”), to a composition for preventing cirrhosis (hereinafter may be referred to as a “cirrhosis-preventing composition”), and to a composition for preventing hepatocellular carcinoma (hereinafter may be referred to as a “hepatocellular carcinoma-preventing composition”).

### BACKGROUND ART

Since old days, mushrooms have been frequently used as food materials having unique flavors and tastes. Having physiological function activating actions, such as enhancement of immunocompetence, antimicrobial activity, control of biorhythm, and prevention of senescence, mushrooms have also been used as Chinese herbal medicines or folk medicines for certain types of diseases. Studies of pharmacological ingredients concerned with mushrooms are in progress, resulting in the discovery of ingredients exerting antibacterial and antiviral actions, a cardiotonic action, a hypoglycemic action, a cholesterol-lowering action, an anti-thrombotic action, and an anti-hypertensive action.

The present applicant previously found a novel fungus Basidiomycetes-X FERM BP-10011 (hereinafter referred to simply as “Basidiomycetes-X”) and filed a patent application on an extract composition thereof (hereinafter referred to as a “Basidiomycetes-X extract composition”) (see Patent Document 1). The Basidiomycetes-X extract composition, containing a large amount of polysaccharide (β-D-glucan), exhibits high anti-oxidative power and OH radical-scavenging action. Thus, the composition is expected to exhibit an anti-aging action and the like. The Basidiomycetes-X extract composition, having an immunomodulating action, is suitably used as an immunoactivator or the like. Also, the present applicant previously filed a patent application on a composition for ameliorating/preventing an atopic disease, which composition is based on the Basidiomycetes-X extract composition (see Patent Document 2).

Meanwhile, in a global trend for westernization of dietary life in recent years, the amount of fat-rich food taken increases along with an increase in stress level or the like, whereas the amount of exercise decreases under recent social circumstances. This tendency becomes more and more significant, and the levels of fat accumulated in human bodies have risen critically, causing a social problem. Fat and lipid taken into a body in excessive amounts are accumulated on various tissues, conceivably triggering lifestyle-related diseases in many cases. For example, anomalous cytokine secretion in adipocytes of an internal organ which has been enlarged through accumulation of excessive fat is a main cause for metabolic syndromes including diabetes or arteriosclerosis. Also, the amount of fat which exceeds the retention limit of adipocytes causes inflammation in the relevant internal organ. In the case of the liver, a fatty liver disease or the like occurs.

Regarding fatty liver diseases, there are many problematic cases in which onset of a fatty liver disease is observed in subjects without drinking history or with only a short drinking history (≥20 g/day for women, ≥30 g/day for men). Such a case is called non-alcoholic fatty liver disease (NAFLD), which is broadly divided into simple steatosis and non-alcoholic steatohepatitis (NASH). NASH is based on simple steatosis concomitant with inflammation and fibrosis and is thought to be a disease of poor prognosis (see Non-Patent Document 1). Of these, in some cases, NASH may be altered to cirrhosis or hepatocellular carcinoma. Thus, taking countermeasures against NASH is a critical and urgent issue.

Since NASH is considered to be a type of metabolic syndrome, symptoms of NASH accompany a lifestyle-related disease such as obesity, diabetes, hyperlipemia, or hypertension. It is inferred that, in European countries and the US, 20% to 30% of the population suffer steatosis, and about 3% of the population develop NASH. In addition, the number of obese subjects and lifestyle-related disease subjects has also increases in countries other than European countries and the US. Thus, a worldwide and rapid increase in the number of steatohepatitis subjects is anticipated.

The clinical manifestation of NASH mainly includes a rise in transaminase activity (mainly due to alanine transaminase (ALT)) and a fibrosis marker (e.g., hyaluronic acid level) level. The diagnosis of NASH requires hepatobiopsy for checking pathological findings which include deposition of fat droplets, infiltration of inflammatory cells, hepatic fibrosis, and formation of ballooning hepatocyte, thereby making the diagnosis difficult. Furthermore, in a case resulting in cirrhosis (i.e., burn out NASH), fat droplets disappear, thereby making the diagnosis more difficult.

Therefore, in some cases, NASH is determined through exclusion of other diseases, and early diagnosis is impeded. Thus, NASH progresses to a fatal disease such as cirrhosis or hepatocellular carcinoma, before the patient receives an appropriate treatment for amelioration. Currently, 30% of the patients diagnosed with NASH suffer cirrhosis after a lapse of 10 years, and half of the cirrhosis patients suffer hepatic failure.

There has not been developed an NAFLD- or NASH-ameliorating medicine whose efficacy has been definitely confirmed. As a result, reducing body weight through improvement of eating habits and/or therapeutic exercises is the first choice for the amelioration thereof. In some cases, medication targeting lifestyle-related diseases possibly causing NASH may be carried out in parallel, wherein a medicine such as an insulin resistance improving agent, an anti-oxidizing agent (e.g., vitamin E), a liver supporting agent, or an angiotensin II receptor antagonist is employed. Although such medication is effective, it is often avoided from the viewpoint of adverse side effects caused by long-term administration. Thus, efforts are made to explore, rather than medication, a high-safety therapeutic strategy employing food or a natural product which has been eaten for a long period of time.

### PRIOR ART DOCUMENTS

### Patent Documents

Patent Document 1: WO 2004/097007

Patent Document 2: Japanese Patent Application Laid-Open (kokai) No. 2007-109449

### Non-Patent Documents

Non-Patent Document 1: Naoki Tanaka, et al., “Liver,” 2002, Vol. 43, No. 12, p. 539-549

### SUMMARY OF THE INVENTION

### Problems to be Solved by the Invention

However, none of Patent Documents 1 and 2 and Non-Patent Document 1 discloses that Basidiomycetes-X is applied to amelioration nonalcoholic steatohepatitis (hereinafter abbreviated as NASH) or preparation of a food composition or a beverage composition which can prevent NASH; or that Basidiomycetes-X can prevent aggravation of NASH to cirrhosis or hepatocellular carcinoma. These documents are silent to such effects of Basidiomycetes-X, which the present inventors have proven.

Under such circumstances, an object of the present invention is to provide, by using Basidiomycetes-X FERM BP-10011, which is highly safe and easy to be taken perorally, an anti-NASH composition, a NASH-preventing food composition, a NASH-preventing beverage composition, a cirrhosis-preventing composition, and a hepatocellular carcinoma-preventing composition.

### Means for Solving the Problems

In order to attain the aforementioned object, the present inventors have conducted extensive studies, and have found that Basidiomycetes-X FERM BP-10011 has high safety; can be processed into a form for easy oral ingestion; ameliorates NASH; and prevents transition of NASH to cirrhosis or hepatocellular carcinoma. The present invention has been accomplished on the basis of this finding.

In a first mode of the present invention to attain the aforementioned object, there is provided an anti-NASH composition comprising, as an active ingredient, a Basidiomycetes-X FERM BP-10011 dry powder or an extract composition thereof.

A second mode of the present invention is a specific embodiment of the anti-NASH composition of the first mode, which is in the form selected from among powder, granule, tablet, capsule, solution, and gel.

In a third mode of the present invention, there is provided a NASH-preventing food composition comprising, as an active ingredient, a Basidiomycetes-X FERM BP-10011 dry powder or an extract composition thereof.

In a fourth mode of the present invention, there is provided a NASH-preventing beverage composition comprising, as an active ingredient, a Basidiomycetes-X FERM BP-10011 dry powder or an extract composition thereof.

In a fifth mode of the present invention, there is provided a cirrhosis-preventing composition which comprises, as an active ingredient, a Basidiomycetes-X FERM BP-10011 dry powder or an extract composition thereof and which prevents transition of NASH to cirrhosis.

In a sixth mode of the present invention, there is provided a hepatocellular carcinoma-preventing composition which comprises, as an active ingredient, a Basidiomycetes-X FERM BP-10011 dry powder or an extract composition thereof and which prevents transition of NASH to hepatocellular carcinoma.

### Effects of the Invention

The present invention employs Basidiomycetes-X, which is highly safe and easy to be taken perorally, to thereby provide an anti-NASH composition, a NASH-preventing food composition, a NASH-preventing beverage composition, a cirrhosis-preventing composition, and a hepatocellular carcinoma-preventing composition.

### BRIEF DESCRIPTION OF THE DRAWINGS

FIG. 1 Graphs (a) to (e) show the results of blood tests of the test groups ((a): ALT level, (b): AST level, (c): APL level, (d): TC level, and (e): TG level).

FIG. 2 Graphs (a) to (c) show measurements of organ weight and blood glucose level of the test groups ((a): liver weight/body weight, (b): spleen weight/body weight, and (c): blood glucose level).

FIG. 3 Photographs (a) to (l) of the tissues of the test groups ((a) to (d): liver images, (e) to (h): liver tissue images stained with H&E, and (i) to (l): images of fibrosis area stained with MT).

FIG. 4 Graphs (a) to (c) show the measurements of protein expression amounts of the test groups obtained through Western blotting ((a): PPARα/GAPDH, (b): PPARγ/GAPDH, and (c): Cytochrome C/GAPDH).

FIG. 5 Graphs (a) and (b) show the measurements of protein expression amounts of the test groups obtained through Western blotting ((a): SIRT1/GAPDH and (b): Glut4/GAPDH).

FIG. 6 Graphs (a) to (c) show the measurements of protein expression amounts of the test groups obtained through Western blotting ((a): p-NF-κB/NF-κB, (b): IL-1β/GAPDH, and (c): IL-10/GAPDH).

### MODES FOR CARRYING OUT THE INVENTION

The anti-NASH composition of the present invention contains, as an active ingredient, a Basidiomycetes-X dry powder or an extract composition thereof.

As used herein, the term “Basidiomycetes” refers to a basidiomycete characterized by having beaklike protrusions (i.e., clamps) but no basidium-formability, differing from other basidiomycetes. That is, even when the basidiomycete of the present invention is cultured, only sclerotia (hypha masses) are formed, but the basidia are not formed. Such a basidiomycete was obtained through retrieving a fungus from the natural world. The basidiomycete is isolated and deposited as “Basidiomycetes-X” to the NITE International Patent Organism Depositary (NITE-IPOD) of the National Institute of Technology and Evaluation (NITE) (Accession Number: FERM BP-10011).

The Basidiomycetes-X forms no conidia, or has no asexual generation. For example, when the Basidiomycetes-X is cultured in a potato glucose agar medium, the hyphae (or mycelia) formed through culturing are smooth and have clamps, but form no conidium or fruit body. Through observation of the morphology and color tone of the colony surface, light pinkish hypha masses are formed. In the case where a plurality of hypha masses are formed in a colony concentrically grown from the inoculation site, the hypha masses are interconnected via mycelial strands. Notably, the backside of the colony assumes light pink. When the Basidiomycetes-X is cultured in a glucose-dry yeast agar medium, the hyphae formed through culturing are smooth and have clamps, but form no conidium or fruit body. Through observation of the morphology and color tone of the colony surface, “light pink to white” hypha masses are formed. Hypha masses having a thickness of 5 mm to 6 mm are formed to surround the inoculation site. Notably, the backside of the colony assumes “light pink to white.”

The optimum growth conditions for Basidiomycetes-X include, for example, a pH of 5.0 to 6.0 and a growth temperature of 22° C. to 26° C. The growth allowable conditions include, for example, a pH of 4.0 to 7.5 and a growth temperature of 5° C. to 30° C.

No particular limitation is imposed on the method of culturing the Basidiomycetes-X, and the aforementioned customary method may be employed. In one exemplary mode of culturing, cultured Basidiomycetes-X cells or seed Basidiomycetes-X cells are aseptically inoculated to an agar medium, a sawdust medium, a liquid medium, or the like to which appropriate nutrient sources have been added and which has been sterilized. Culturing is performed at a suitable temperature, whereby hypha masses of the Basidiomycetes-X can be yielded. Notably, the Basidiomycetes-X forms various hypha masses depending on the culture circumstances.

If needed, the thus-formed hypha masses of Basidiomycetes-X are dried, and the dry product is pulverized, to thereby yield a Basidiomycetes-X dry powder, which is an embodiment of the anti-NASH composition of the present invention. Alternatively, the dry powder may be formed into an anti-NASH composition having a shape of granule, tablet, capsule, solution, gel, etc.

Alternatively, a Basidiomycetes-X extract composition may be provided as an active ingredient of the anti-NASH composition of the present invention. No particular limitation is imposed on the extracting an active ingredient from Basidiomycetes-X hypha masses. In one mode of efficiently extracting cell contents from Basidiomycetes-X hypha masses, preferably, cell walls of the Basidiomycetes-X hypha masses are optionally broken through, for example, freezing. The product is thawed and broken by means of a mixer or the like, and an extract (i.e., Basidiomycetes-X extract composition) is yielded.

No particular limitation is also imposed on the solvent for use in extraction, and water, a lower alcohol, etc. may be used. Also, an extraction solvent further containing an acid, an alkali, or another additive may be used. Extraction is performed at ambient temperature or under heating or pressure. In one general mode of extraction, Basidiomycetes-X hypha masses are boiled in water for extraction. In an alternative mode, a broken product of Basidiomycetes-X hypha masses is mixed with water or an aqueous mixture containing an alcohol or an alkali, and the resultant mixture is pressurized at, for example, about 100 MPa to about 700 MPa, preferably 300 MPa to about 600 MPa, for extraction.

An example of the extraction method will next be described. Firstly, frozen Basidiomycetes-X hypha masses are thawed at ambient temperature and broken by means of a mixer. The ratio in amount of the broken Basidiomycetes-X hypha mass to that of water (extraction solvent) is adjusted to, for example, about 1:5. Specifically, the broken Basidiomycetes-X hypha masses (50 g) are placed in a glass bottle, and water (250 mL) is added to the bottle. The bottle is closed. Separately, a towel is placed on the bottom of a pan, and water is poured onto the towel. The glass bottle accommodating the broken product of the Basidiomycetes-X hypha masses is mounted on the towel, and the pan is heated to boil water. Heating is continued for 90 minutes after boiling, and the contents of the glass bottle are cooled. Through phase separation, an extract (Basidiomycetes-X extract composition) and a residue (Basidiomycetes-X extraction residue) are yielded. The pH of the extract is, for example, 6.3 to 6.5. Instead of a broken product of Basidiomycetes-X hypha masses, a Basidiomycetes-X dry powder may be used. In this case, the Basidiomycetes-X dry powder is statically cultured in an aqueous medium for 4 hours to 6 hours, while the medium is suitably stirred. The product is subjected to phase separation, to thereby yield an extract and a residue (Basidiomycetes-X extraction residue).

The thus-obtained extract is optionally concentrated, to thereby provide a Basidiomycetes-X extract composition. No particular limitation is imposed on the extract concentration method, and one exemplary mode is as follows.

Firstly, the obtained extract is transferred to a beaker and is concentrated through heating and evaporation. In the course of concentration, the color of the extract changes from light beige to brown, and vigorous effervescence is observed. Evaporation/concentration is further performed. When the extract assumes a tar-like liquid having a pH of 4.9 and a density of 1.25 g/cm³, concentration is stopped. The thus-concentrated extract gives off a soy source-like flavor. At this timing, the average yield of the concentrated extract from the Basidiomycetes-X hypha masses is 12%. Since the viscosity of the thus-obtained concentrated extract steeply increases during cooling of the extract, the extract must be transferred to a storage container immediately after termination of concentration. After cooling, the concentrated extract placed in the storage container is preferably stored in a frozen state.

The thus-obtained Basidiomycetes-X extract composition is optionally dried and processed into a form of powder, granule, tablet, capsule, solution, gel, etc., to thereby provide the anti-NASH composition of the present invention. Alternatively, the anti-NASH composition of the present invention may be a Basidiomycetes-X dry powder. No particular limitation is imposed on the amount of the anti-NASH composition in each of the above products, and the amount may be suitably tuned.

The anti-NASH composition of the present invention may provide the NASH-preventing food composition or the NASH-preventing beverage composition, having any form selected from among powder, granule, tablet, capsule, solution, gel, etc. Through further optional processing of any of the compositions, a supplement, a beverage, and the like may be provided. No particular limitation is imposed on the amount of the Basidiomycetes-X dry powder or the Basidiomycetes-X extract composition in each of the NASH-preventing food composition and the NASH-preventing beverage composition, and the amount may be suitably tuned.

As described in the Examples, the anti-NASH composition of the present invention can ameliorate NASH. Also, the cirrhosis-preventing composition and the hepatocellular carcinoma-preventing composition can prevent transition of NASH to cirrhosis or hepatocellular carcinoma. Thus, NASH can be ameliorated by administering the anti-NASH composition of the present invention to a patient in need thereof. Also, through administering, to a patient in need thereof, the cirrhosis-preventing composition or the hepatocellular carcinoma-preventing composition, transition of NASH to cirrhosis or hepatocellular carcinoma can be prevented.

Also, the anti-NASH composition of the present invention can be used for preventing and treating NASH. The cirrhosis-preventing composition or the hepatocellular carcinoma-preventing composition may be used for preventing cirrhosis or hepatocellular carcinoma. In such NASH-treating method, cirrhosis-preventing method, and hepatocellular carcinoma-preventing method, no particular limitation is imposed on the method of causing a patient to take each composition, and the effective amount of the composition may be appropriately determined depending on the extent of NASH, the symptoms attributed to NASH, and other factors. The patient may take the composition in the thus-determined amount. In the present embodiment, oral ingestion is preferred, from the viewpoint of easiness in daily life. In the NASH-treating method, cirrhosis-preventing method, and hepatocellular carcinoma-preventing method, one exemplary mode of oral ingestion includes causing a patient to take a Basidiomycetes-X extract composition dry powder preferably charged into capsules each having a dose of 200 mg to 300 mg, one to thrice per day, preferably thrice per day. No particular limitation is imposed on the ingestion period, but the period is preferably long, for example, preferably 8 weeks or longer, more preferably 16 weeks or longer. Alternatively, the Basidiomycetes-X dry powder may be in the form of tablet or liquid such as syrup, for ingestion.

### EXAMPLES

The present invention will next be described in more detail by way of the Examples and the Production Examples of Basidiomycetes-X dry powder and Basidiomycetes-X extract composition. Notably, Production Examples 1 to 4 are directed to culturing of Basidiomycetes-X; Production Examples 5 is directed to drying of Basidiomycetes-X; and Production Example 6 is directed to production of Basidiomycetes-X extract composition dry powder.

### Production Example 1

### Separation from Hypha Masses

### (1) Preparation of Culture Medium

A PSA medium and a PDA medium having the compositions shown in Table 1 were prepared. Each medium was dispensed into a test tube or an Erlenmeyer flask, which was stoppered with Silicosen (or a cotton plug). These media were sterilized with high-pressure vapor at 121° C. for 20 minutes in an autoclave. In the case of a test tube, a hot medium after sterilization was formed into a slant medium, whereas in the case of an Erlenmeyer flask, a sterilized medium was allowed to stand to form a plate medium.

TABLE 1

| PSA medium | PDA medium |
|---|---|
| Petro 200 g | Petro 200 g |
| (20 min- boil/extract) | (20 min- boil/extract) |
| Sucrose 20 g | Glucose 20 g |
| Agar 15 g | Agar 15 g |
| Total volume 1 L | Total volume 1 L |

### (2) Separation from Hypha Masses

Larger Basidiomycetes-X hypha masses were broken manually, and slices were cut from Basidiomycetes-X sections with a scalpel which had been flame-sterilized and cooled. The PSA medium and the PDA slant medium of (1) were each inoculated with the Basidiomycetes-X slices by means of tweezers which had been flame-sterilized and cooled. This procedure was performed under aseptic conditions in an aseptic box or a clean bench.

### (3) Culturing in Agar Medium for Production of Hypha Masses

Potato dices (1 cm×1 cm) (200 g) were boiled in purified water for 20 minutes and then cooled. The broth was separated from the solid. To a mixture of the broth (potato extract), sucrose (20 g), and agar (1 g, 0.1%), distilled water was added, so that the total volume was adjusted to 1 L, to thereby prepare an agar medium. Although a conventional agar medium has an agar concentration of 1.5 to 2.0 (i.e., 15 g to 20 g based on 1 L of the medium), the agar concentration of this medium was adjusted 0.1%, for facilitating isolation of cultured hypha masses from the agar medium and maintaining the physical strength of Basidiomycetes-X slices, which readily cause sedimentation in a liquid culture medium. The 0.1% agar medium (each 5 mL) was dispensed into test tubes, which were stoppered with Silicosen. These media were sterilized with high-pressure vapor at 121° C. for 20 minutes in an autoclave. Thereafter, a slice was cut from the Basidiomycetes-X hypha masses in culturing on the slant medium of Production Example 1. This operation was performed in an aseptic box after aseptic treatment. The slice was inoculated to the 0.1% agar medium. The inoculum was cultured in an incubator at 24° C., and was found to generate the organism in 24 to 48 hours. After generation of the organism, culture was continued at 24° C. As a result, hyphae grew on the agar media in 14 days.

### Production Example 2

### Culturing in Sawdust Medium for Production of Hypha Mass

### (1) Culturing of Seed Fungus

Water was added to sawdust (1 L), defatted bran (15 g), wheat bran (15 g), and SANPEARL (hypha activator, product of Nippon Paper Industries) (5 g), and the mixture was vigorously stirred. This mixture for culture was adjusted such that when it was firmly gripped, water exuded (water content of the mixture: about 70%), whereby a sawdust medium was prepared. This culture medium was placed in an Erlenmeyer flask, which was stoppered with Silicosen. Then, the Erlenmeyer flask was subjected to high pressure steam sterilization in an autoclave for 40 minutes at 121° C. Twenty-four hours after the sterilization, Basidiomycetes-X hyphae during culture on the slant media in Production Example 1 were inoculated into the sawdust medium within an aseptic box through an aseptic operation. The inoculation was carried out such that no damage was caused to the hyphae, with a sterilized triangular knife being used to cut off a part of the slant medium. The density of the inoculation was adjusted to 20% to 30% of the surface area of the sawdust medium. When the inoculum was cultured at 24° C., the organism was generated in 3 days (in 5 days at the latest). After a lapse of 30 days, the sawdust medium in the Erlenmeyer flask was full of hyphae.

### (2) Generation of Hypha Mass

A sawdust medium was prepared in the same manner as employed in (1). This culture medium was placed in a polypropylene bottle, which was stoppered, and subjected to high pressure steam sterilization in an autoclave for 40 minutes at 121° C. Twenty-four hours after the sterilization, the seed organism cultured in (1) was inoculated into the sawdust medium placed in the polypropylene bottle through an aseptic operation within an aseptic box after aseptic treatment. The density of the inoculation was adjusted such that the surface area of the sawdust medium was virtually covered with the inoculum. When the inoculum was cultured at 24° C., the organism was generated in 48 hours. After a lapse of 60 days, the entire sawdust medium within the polypropylene bottle was full of hyphae. After a further lapse of 40 to 50 days, hyphae spread on the inner wall of the polypropylene bottle, forming mycelial strands. When culture was continued further, hypha masses were formed.

### Production Example 3

### Production of Basidiomycetes-X Dry Powder

In order to cause damage to the cell walls of the hyphae and facilitate the leaching-out of the cell contents, fresh Basidiomycetes-X hypha masses obtained in Production Example 2 were frozen. The thus-frozen Basidiomycetes-X hypha masses were thawed at ambient temperature, and crushed by means of a mixer. The product is dried to form a powder (hereinafter referred to as “Basidiomycetes-X dry powder”).

### Production Example 4

### Production of Basidiomycetes-X Extract Composition Dry Powder

The Basidiomycetes-X dry powder obtained in Production Example 3 was weighed for 4 kg (dry weight). Water (20 L) was added to the dry powder, and the mixture was subjected to static culturing for 4 to 6 hours under appropriate stirring. Subsequently, the solid contents (hereinafter referred to as “Basidiomycetes-X extraction residue”) in the culture were removed through vacuum filtration, to thereby yield 17.6 kg of a Basidiomycetes-X extract composition (solid content: 8.0%). Then, the product was preliminarily frozen at −40° C. and lyophilized (hereinafter the product being referred to as “Basidiomycetes-X extract composition dry powder”).

### Example 1

The Basidiomycetes-X extract composition dry powder obtained in Production Example 4 was dissolved in water so that the daily dose was regulated to 500 mg/kg-body weight. The solution was tested.

### (1) Tested Animals and NASH Treatment Method through Administration of Basidiomycetes-X

C57BL/6 female mice (soon after birth) were divided into the following four groups: a healthy (normal) subject group without onset of non-alcoholic steatohepatitis (NASH) (n=5) (hereinafter referred to as “normal group”); a non-treatment group with onset of light NASH (n=5) (hereinafter referred to as “HFD-8W group”); a non-treatment group with onset of heavy NASH (n=8) (hereinafter referred to as “NASH group”); and a NASH-amelioration group with onset of heavy NASH and administration of 5% Basidiomycetes-X extract composition dry powder (n=6) (hereinafter referred to as “NASH+mushroom group”).

### (2) Induction of NASH

Except the healthy group, streptozotocin (STZ) (200 μg) was hypodermically injected to each mouse of about 1 week old. All the mice of the four groups were preliminarily bred with a normal diet for 4 weeks after birth. Then, the mice were offered a healthy or a high-fat feed (product of CLEA Japan, Inc.) in the following manner.

In the normal group, mice were bred with ad libitum feeding of a normal diet over 12 weeks from week 4. In the HFD-8W group, the normal diet was altered to a high-fat feed in week 4, and mice were bred with ad libitum feeding of the high-fat diet over 8 weeks from the change of feed. In the NASH group, the normal diet was altered to a high-fat feed, and mice were bred with ad libitum feeding of the high-fat diet over 12 weeks from the change of feed. In the NASH+mushroom group, the normal diet was altered to a high-fat feed in week 4, and mice were bred with ad libitum feeding of the high-fat diet over 12 weeks from the change of feed. In the NASH+mushroom group, the Basidiomycetes-X extract composition dry powder dissolved in water (i.e., a test substance) was administered to mice for 5 weeks (week 12 to week 16 after birth). The test substance was orally administered once a day by means of a probe so that the daily dose was adjusted to 500 mg/kg-body weight.

### (3) Blood Test

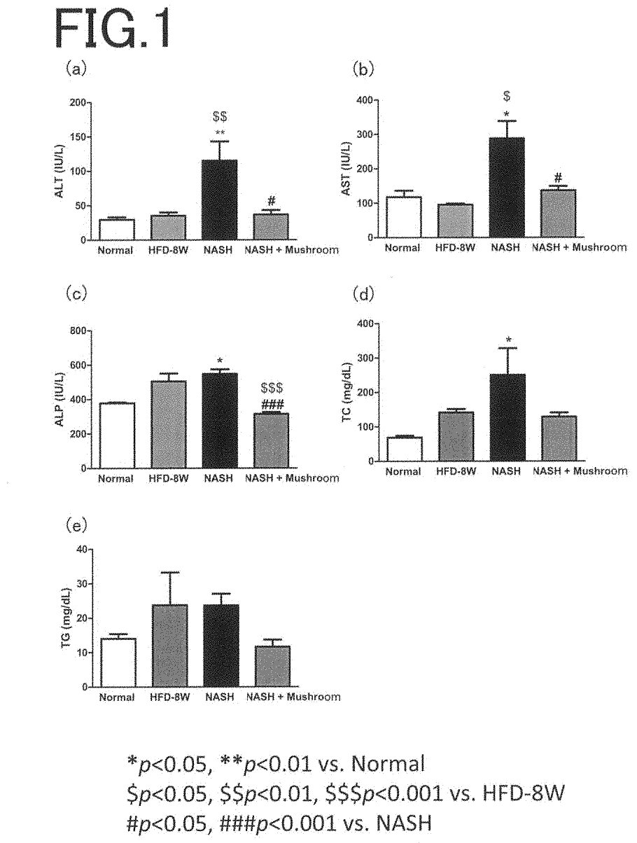

After passage of 12 weeks or 16 weeks, the mice of the four groups were fasted overnight, and blood was collected from the mice after fasting. The blood samples were tested, and FIG. 1 shows the results. In FIG. 1, graphs (a) to (e) show the results of the blood tests ((a): ALT level, (b): AST level, (c): APL level, (d): TC level, and (e): TG level).

The measurement items; aspartate transaminase (AST), alanine aminotransferase (ALT), and alkaline phosphatase (ALP) are present in the liver tissue. When hepatocytes are damaged, these enzymes are discharged from the cells (i.e., deviation enzymes). Thus, the level of such an enzyme is a key index for assessing the liver function. In addition to the three enzyme levels, total cholesterol (TC) and triglyceride (TG) were measured.

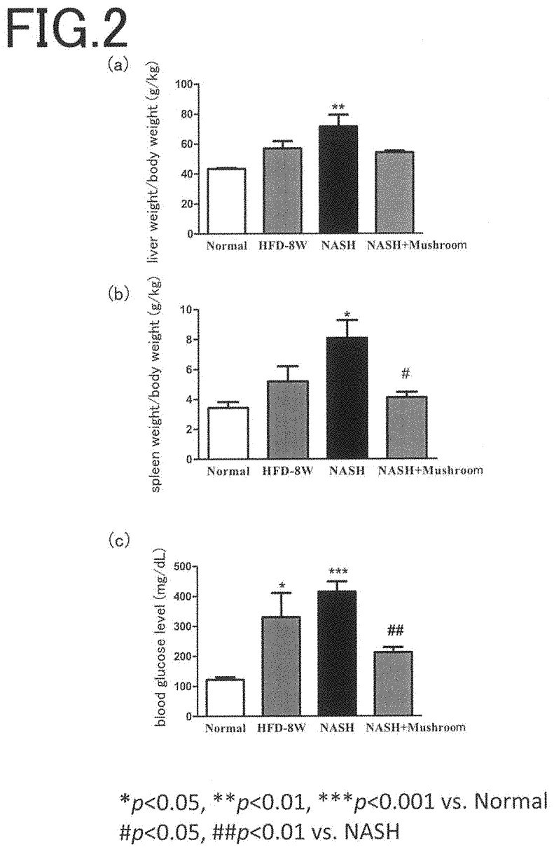

Each of the numerical values in FIG. 1 is represented by “average±standard deviation.” Statistical analysis was performed through one-way analysis of variance (one-way ANOVA, followed by Dunnett's method), and significance was assessed by a P value of <0.05. In the below-described tests (see FIG. 2 and FIGS. 4 to 6), the statistical processing was performed by the same analysis.

As shown in FIG. 1, the liver function parameters (the ALT, AST, and ALP levels) of the NASH+mushroom group were significantly reduced, as compared with those of the NASH group. The TG and TC levels of the NASH+mushroom group were generally lower than those of the NASH group.

### (4) Measurement of Organ Weight and Blood Glucose Level

After completion of the tests (1) above, the mice were sacrificed and dissected. For each mouse of the test groups, the liver weight (LW) and the spleen weight (Sp) with respect to the body weight (BW) were measured, along with the blood glucose level. The results are shown in FIG. 2. In FIG. 2, graphs (a) to (c) show the measurements of organ weights and blood glucose level of the test groups ((a): liver weight/body weight, (b): spleen weight/body weight, and (c): blood glucose level).

As is clear from FIG. 2(a), the liver weight/body weight (hereinafter referred to as “LW/BW”) of the NASH+mushroom group is generally reduced, as compared with that of the NASH group. Also, as shown in FIGS. 2(b) and 2(c), the spleen weight/body weight (hereinafter referred to as “Sp/BW”) and the blood glucose level of the NASH+mushroom group are significantly reduced, as compared with those of the NASH group. Thus, the tests have clearly revealed that an increase in Sp/BW and blood glucose level can be suppressed through ingestion of Basidiomycetes-X extract composition dry powder. The tendency of reduction in liver weight suggests amelioration of enlargement of the liver, and the normalization of the spleen weight suggests improvement of the immune system increase.

### (5) Tissue Observation

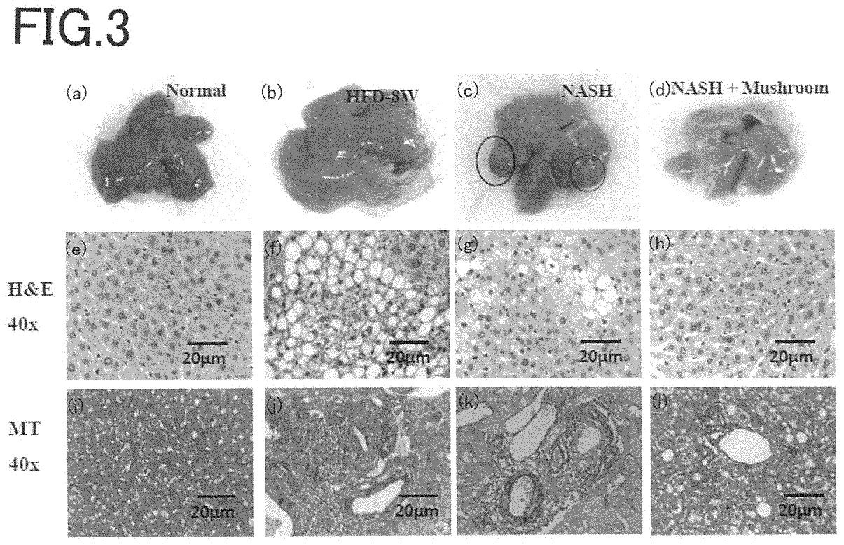

In dissection conducted in (4) above, the liver was removed from each mouse. The liver tissue was stained by hematoxylin eosin (hereinafter referred to as “H&E stain”) and by Masson trichrome (hereinafter referred to as “MT” stain). FIG. 3 shows the results of observation.

In FIGS. 3, (a) to (l) are photographs of the tissues of the test groups ((a) to (d): liver images, (e) to (h): liver images stained with H&E, and (i) to (l): images of fibrosis area stained with MT).

According to the liver images of the test groups, the liver of the NASH+mushroom group shown in FIG. 3(d) was somewhat similar in appearance to the liver of the normal group shown in FIG. 3(a). That is, pathological conditions characteristic to NASH, such as deposition of fat droplets, inflammation, formation of ballooning hepatocyte, and formation of hepatocellular carcinoma, were found to be suppressed. In contrast, the liver of the HFD-8W group shown in FIG. 3(b) exhibited steatosis. The liver of the NASH group shown in FIG. 3(c) clearly exhibited pathological conditions characteristic to NASH, including formation of ballooning hepatocyte and formation of hepatocellular carcinoma, particularly in a circled area.

Also, as shown in FIGS. 3(e) to 3(h), the state of the liver of the NASH+mushroom group was somewhat similar in appearance to the liver of the normal group, as confirmed in the H&E stain images. That is, pathological conditions characteristic to NASH, such as deposition of fat droplets, infiltration of inflammation cells, formation of ballooning hepatocyte, and formation of hepatocellular carcinoma, were found to be suppressed. In contrast, the liver of the HFD-8W group exhibited steatosis. The liver of the NASH group gave a liver tissue image considerably exhibiting the aforementioned conditions attributed NASH. Particularly, in case of the NASH group as shown in FIG. 3(g), some areas where fat droplets had disappeared were observed. The liver image indicated that the case was conceivably aggravated to cirrhosis (i.e., burn out NASH). Notably, since fat cannot be stained through H&E staining, the white (quasi-transparent) areas of the photoimage correspond to those of fat.

Furthermore, as shown in FIGS. 3(i) to 3(l), significant suppression of fibrosis of the liver of the NASH+mushroom group was confirmed by the results of MT staining. The liver state was almost ameliorated to the state of the normal. However, in the liver slices of the HFD-8W and NASH groups, irreversible fibrosis was observed. Particularly, in the liver of the NASH group shown in FIG. 3(k), significant liver fibrosis was observed.

As shown in FIGS. 3(a) to 3(l), the tests have indicated that Basidiomycetes-X can suppress or ameliorate pathological conditions characteristic to NASH, such as deposition of fat droplets, infiltration of inflammation cells, liver fibrosis, formation of ballooning hepatocyte, and formation of hepatocellular carcinoma. In other words, it is strongly suggested that the Basidiomycetes-X can prevent transition of NASH to cirrhosis or hepatocellular carcinoma.

### (6) Western Blotting

The liver tissue obtained through dissection performed in (2) above was treated by means of Polytron, and the protein content of the thus-homogenized product was determined through the bicinchoninic acid (BCA) method. Thereafter, a sample buffer added twice to the homogenized product, to thereby prepare samples for Western blotting. Each sample was subjected to electrophoresis by use of a 10% SDS-polyaclylamide gel electrophoresis (SDS-PAGE) gel at 150 V for 50 minutes, and all the proteins present on the gel were transferred to a nitrocellulose membrane at 10 V for 60 minutes. After completion of transfer, bands of the membrane were visualized by staining with Poncean S and washed with PBS. The membrane was blocked with 5% BSA for 1 hour.

The membrane was reacted overnight with primary antibody at 4° C. in a refrigerator. The antibodies were peroxisome proliferator-activated receptor (PPAR) α (1:1000), PPARγ (1:1000), cytochrome C (cyt c) (1:1000), sirtuin (SIRT)-1 (1:1000), glucose transporter type 4 (Glut4) (1:1000), nuclear factor-κB (NF-κB) (1:1000), phospho-NFκB (p-NF-κB) (1:1000), interleukin-1β (IL-1β) (1:1000), and (interleukin-1β:IL-10 (IL-10) (1:1000). As an internal standard, glyceraldehyde-3-phosphate-dehydrogenase (GAPDH) (1:8000) was used.

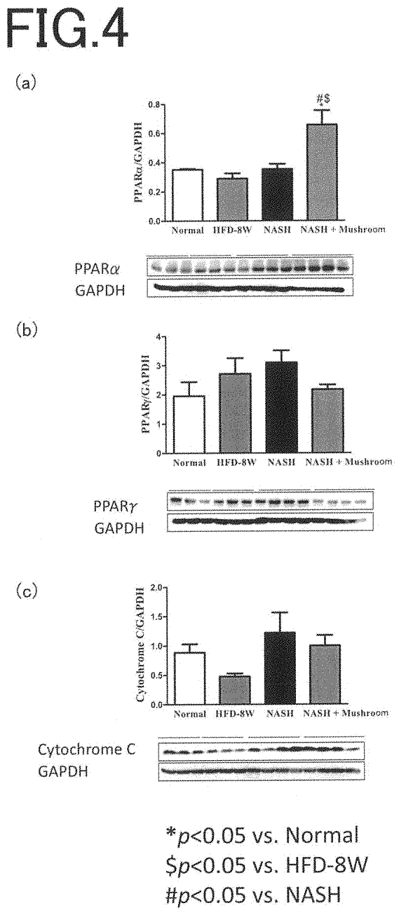

On the next day, the membrane was washed once with TBS-Tween 20 and then reacted with a secondary antibody: an anti-rabbit antibody (1:10000), an anti-mouse antibody (1:10000), or an anti-goat antibody (1:10000) at room temperature for 1 hour. The amount of each protein formed was determined by use of ImmunoStar LD and by means of C-DiGit blot scanner (M&S TechnoSystems, Inc.). The p-NF-κB expression amount was divided by the corresponding NF-κB expression amount, and each of the PPARα, PPARγ, Cytochrome C, SIRT1, Glut4, IL-1β, and IL-10 expression amounts was divided by the corresponding GAPDH expression amount. These expression amounts were compared between test groups. The results are shown in FIGS. 4 to 6.

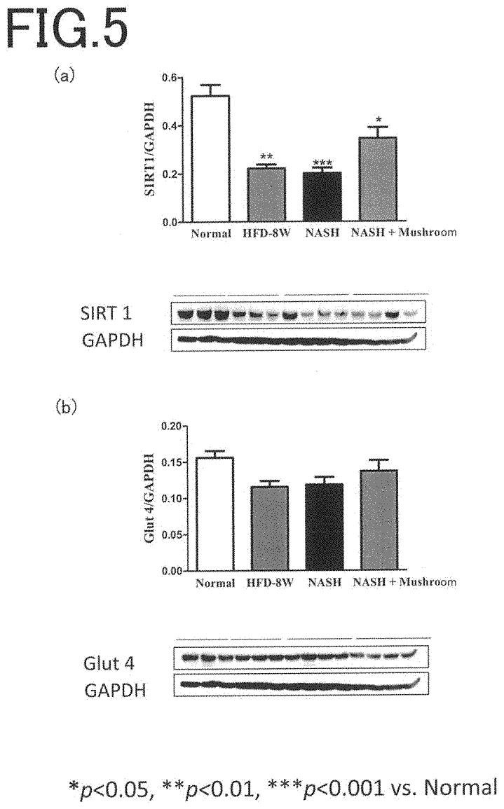

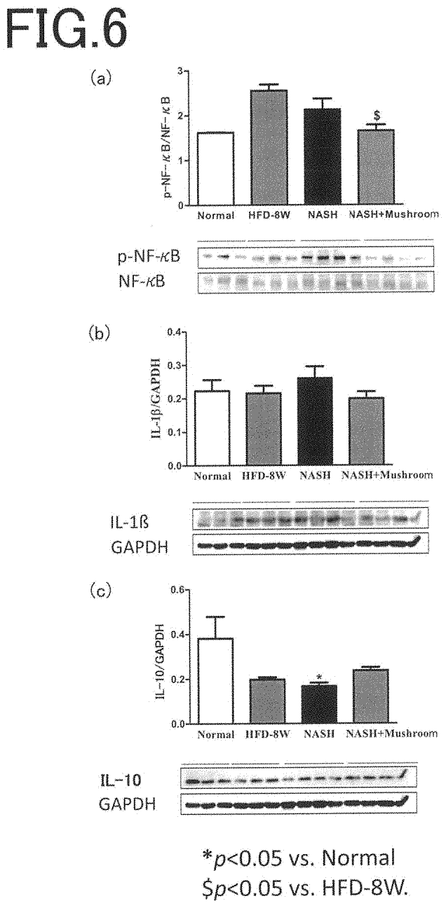

In FIG. 4, graphs (a) to (c) show the measurements of protein expression amounts of the test groups obtained through Western blotting ((a): PPARα/GAPDH, (b): PPARγ/GAPDH, and (c): Cytochrome C/GAPDH). In FIG. 5, graphs (a) and (b) show the measurements of protein expression amounts of the test groups obtained through Western blotting ((a): SIRT1/GAPDH and (b): Glut4/GAPDH). In FIG. 6, graphs (a) to (c) show the measurements of protein expression amounts of the test groups obtained through Western blotting ((a): p-NF-κB/NF-κB, (b): IL-1β/GAPDH, and (c): IL-10/GAPDH).

PPAR is a nuclear receptor belonging to the steroid hormone receptor superfamily and has three subtypes: α, β, and γ. PPARα is predominantly present in organs where oxidation of fatty acid actively occurs, particularly in the liver, the heart, the digestive tract, etc. In the case of the liver, it is known that proliferation of peroxisomes via activation of PPAR can rapidly and drastically change the states of β oxidation of ultra-long-chain fatty acid, synthesis of bile acid, and expression of various genes, enzymatic activity, and metabolism in the liver.

As shown in FIG. 4(a), the PPARα expression amount in the NASH+mushroom group significantly increased, as compared with the expression amounts of the HFD-8W group and the NASH group. This suggests that fat/lipid metabolism including β oxidation of fatty acid and synthesis of bile acid is promoted through administration of Basidiomycetes-X. The results also suggest that administration of Basidiomycetes-X possibly leads to amelioration of blood lipid (TC and TG) shown in FIGS. 1(d) and 1(e).

Meanwhile, formation of PPARγ, which is a protein relating to differentiation of adipocytes, is known to be promoted in the liver of an obesity subject (steatosis) (see Naoki TANAKA, et al., “The Shinshu Medical Journal,” 2008, Vol. 56, No. 6, p. 347-358). As shown in FIG. 4(a), through administration of Basidiomycetes-X, the PPARγ expression amount in the NASH+mushroom group tended to increase, as compared with the expression amounts of the HFD-8W group and the NASH group.

As shown in FIG. 5, the SIRT1 and Glut4 expression amounts in the NASH+mushroom group tended to increase, as compared with the expression amounts of the HFD-8W group and the NASH group. Activation of SIRT1 and Glut4 is known to improve insulin resistance. Thus, it is suggested that the amelioration of blood glucose level in the NASH+mushroom group shown in FIG. 2(c) is attributed to improvement of insulin resistance via activation of SIRT1 and Glut4.

Although still controversial, it is suggested that a certain mushroom possibly serves as a tumor suppressor via activation of the gene repair system (see Hidetaka OHTA, “Japanese Journal of Geriatrics,” 2010, Vol. 47, No. 1, p. 11-16). The report suggests that the tumor suppression somewhat correlates with the present invention; i.e., amelioration of NASH, in particular, a function of preventing transition of NASH to cirrhosis or hepatocellular carcinoma.

As shown in FIG. 6(a), the p-NF-κB/NF-κB expression amount in the NASH+mushroom group was significantly reduced, as compared with that of the HFD-8W group. Since NF-κB closely relates to elicitation of inflammation by serving as a master regulator for inflammation, suppression of NF-κB activation would conceivably lead to amelioration of inflammation in the liver. Also, the aforementioned increase in PPARα level competitively inhibits NF-κB activity, to thereby exhibit anti-inflammatory action. Thus, the NASH-ameliorating effect of the present invention can be elucidated to be based on the anti-inflammatory action via suppression of NF-κB activity, which action is exerted by an increase in PPARα level. As shown in FIGS. 6(b) and 6(c), the IL-1β/GAPDH and the IL-10/GAPDH expression amounts in the NASH+mushroom group tended to decrease, as compared with the normal group. This suggests that the inflammation of the liver was possibly ameliorated.

As described hereinabove, there have been elucidated the actions of oral ingestion of the Basidiomycetes-X extract composition dry powder produced in Example 1 mainly via suppression of the PPARα, the NF-κB, and the SIRT1 expression amounts. The actions include (1) liver tissue repair effect, (2) fat metabolism amelioration effect in liver tissue, (3) hyperglycemia amelioration effect, (4) actions of suppressing deposition of fat droplets, infiltration of inflammatory cells, formation of ballooning hepatocyte, and hepatocellular carcinoma.

### INDUSTRIAL APPLICABILITY

As described herein above, the anti-NASH composition of the present invention is a composition which is derived from a high-safety food or a natural product which has been eaten for a long period of time. The composition can eliminate a severe load such as reducing body weight by changing of a diet style or physical exercise, as well as concerns about adverse side effects due to a long-term administration of a drug targeted to a lifestyle-related diseases caused by NASH. Through ingestion of the anti-NASH composition, amelioration of NASH can be foreseen. The present invention is advantageous, in that the NASH-preventing food composition or the NASH-preventing beverage composition is caused to be taken for a long period of time as a food or a beverage such as a supplement which is acceptable in daily life. Thus, the present invention is safe and simple.

Furthermore, through ingestion of the anti-NASH composition, the cirrhosis-preventing composition, and the hepatocellular carcinoma-preventing composition of the present invention, actions which are attained mainly via suppression of the PPARα, the NF-κB, and the SIRT1 expression amounts are foreseeable. The effects include (1) liver tissue repair effect, (2) fat metabolism amelioration effect in liver tissue, (3) hyperglycemia amelioration effect, (4) actions of suppressing deposition of fat droplets, infiltration of inflammatory cells, formation of ballooning hepatocyte, hepatocellular carcinoma, etc., which are pathological findings intrinsic to NASH. As a result, there can be anticipated an NASH amelioration action, in particular, an action of preventing transition of NASH to cirrhosis or hepatocellular carcinoma.

### Accession Number

### Basidiomycetes-X FERM BP-10011

| | | |
|---|---|---|
| 0-1 | Form PCT/RO/134 | |
| 0-1-1 | The indications (PCT Rule | JPO-PAS i330 |
| | 13bis) made relate to the | |
| | deposited microorganism | |
| | or other biological | |
| | material. | |
| 0-2 | International application No. | |
| 0-3 | Applicant's or agent's | FP20180107 |
| | file reference | |
| 1 | The indications made below | |
| | relate to the microorganism | |
| | or biological material | |
| | referred to in the | |
| | description. | |
| 1-1 | Paragraph | 0024 |
| 1-3 | Indication of deposit | |
| 1-3-1 | Name of depositary | IPOD NITE International Patent |
| | institution | Organism Depositary |
| | | (NITE-IPOD) |
| 1-3-2 | Address of depositary | 120, 2-5-8, Kazusakamatari, |
| | institution | Kisarazu-shi, Chiba 292-0818 |
| | | Japan |
| 1-3-3 | Date of deposit | Feb. 27, 2003 |
| | | (27 Feb. 2003) |
| 1-3-4 | Accession Number | IPOD FERM BP-10011 |
| 1-5 | Designated States for | All designated States |
| | which indications are made | |
| 2 | The indications made below | |
| | relate to the microorganism | |
| | or biological material | |
| | referred to in the description. | |
| 2-1 | Paragraph | 0024 |
| 2-3 | Indication of deposit | |
| 2-3-1 | Name of depositary | IPOD NITE International Patent |
| | institution | Organism Depositary |
| | | (NITE-IPOD) |
| 2-3-2 | Address of depositary | 120, 2-5-8, Kazusakamatari, |
| | institution | Kisarazu-shi, Chiba 292-0818 |
| | | Japan |
| 2-3-3 | Date of deposit | Feb. 27, 2003 |
| | | (27 Feb. 2003) |
| 2-3-4 | Accession Number | IPOD FERM BP-10011 |
| 2-4 | Additional indications | The applicant wishes that the |
| | | biological material shall be |
| | | made available as provided in |
| | | Rule 33(1) EPC only by the issue |
| | | of a sample to an expert |
| | | nominated by the requester. |
| 2-5 | Designated States for | EP: (AL AT BE BG CH&LI CY |
| | which indications are made | CZ DE DK EE ES FI FR GB |
| | | GR HR HU IE IS IT LT LU |
| | | LV MC MK MT NL NO PL PT |
| | | RO RS SE SI SK SM TR) AL |
| | | AT BG CH&LI CZ DE DK EE |
| | | ES FI GB HR HU IS LU MK |
| | | NO PL PT RO RS SE SK SM TR |
| 3 | The indications made below | |
| | relate to the microorganism | |
| | or biological material | |
| | referred to in the description. | |
| 3-1 | Paragraph | 0024 |
| 3-3 | Indication of deposit | |
| 3-3-1 | Name of depositary | IPOD NITE International Patent |
| | institution | Organism Depositary |
| | | (NITE-IPOD) |
| 3-3-2 | Address of depositary | 120, 2-5-8, Kazusakamatari, |
| | institution | Kisarazu-shi, Chiba 292-0818 |
| | | Japan |
| 3-3-3 | Date of deposit | Feb. 27, 2003 |
| | | (27 Feb. 2003) |
| 3-3-4 | Accession Number | IPOD FERM BP-10011 |
| 3-4 | Additional indications | The applicant may request that |
| | | the furnishing of a sample of |
| | | a microorganism shall only be |
| | | made available to an expert. |
| 3-5 | Designated States for which | SG |
| | indications are made | |
| 4 | The indications made below | |
| | relate to the microorganism or | |
| | biological material referred | |
| | to in the description. | |
| 4-1 | Paragraph | 0024 |
| 4-3 | Indication of | |
| | deposit | |
| 4-3-1 | Name of depositary | IPOD NITE International Patent |
| | institution | Organism Depositary |
| | | (NITE-IPOD) |
| 4-3-2 | Address of depositary | 120, 2-5-8, Kazusakamatari, |
| | institution | Kisarazu-shi, Chiba 292-0818 |
| | | Japan |
| 4-3-4 | Accession Number | IPOD FERM BP-10011 |
| 4-4 | Additional indications | The applicant wishes that the |
| | | Commissioner of Patents only |
| | | authorizes the furnishing of |
| | | a sample of deposited biological |
| | | material referred to in the |
| | | application to an independent |
| | | expert nominated by the |
| | | Commissioner. |
| 4-5 | Designated States for which | CA |
| | indications are made | |

1. An anti-NASH composition comprising, as an active ingredient, a Basidiomycetes-X FERM BP-10011 dry powder or an extract composition thereof.

2. An anti-NASH composition according to claim 1, which is in any form selected from among powder, granule, tablet, capsule, solution, and gel.

3. An NASH-preventing food composition comprising, as an active ingredient, a Basidiomycetes-X FERM BP-10011 dry powder or an extract composition thereof.

4. An NASH-preventing beverage composition comprising, as an active ingredient, a Basidiomycetes-X FERM BP-10011 dry powder or an extract composition thereof.

5. A cirrhosis-preventing composition which comprises, as an active ingredient, a Basidiomycetes-X FERM BP-10011 dry powder or an extract composition thereof and which prevents transition of NASH to cirrhosis.

6. A hepatocellular carcinoma-preventing composition which comprises, as an active ingredient, a Basidiomycetes-X FERM BP-10011 dry powder or an extract composition thereof and which prevents transition of NASH to hepatocellular carcinoma.
